(19) United States
(12) Patent Application Publication
(10) Pub. No.: US 2007/0275473 A1
(43) Pub. Date: Nov. 29, 2007

(54) CHEMICAL SENSOR INFERENCE NETWORK FOR FALSE ALARM

(75) Inventor: DAVID C. SWANSON, State College, PA (US)

Correspondence Address: MCKEE, VOORHEES & SEASE, P.L.C.;ATTN: PENNSYLVANIA STATE UNIVERSITY, 801 GRAND AVENUE, SUITE 3200, DES MOINES, IA, 50309-2721, US

(73) Assignee: THE PENN STATE RESEARCH FOUNDATION, University Park, PA (US)

(21) Appl. No.: 11/538,480

(22) Filed: Oct. 4, 2006

Related U.S. Application Data
(60) Provisional application No. 60/723,525, filed on Oct. 4, 2005.

(51) Int. Cl. G01N 33/00

(52) U.S. Cl. 436/104

(57) ABSTRACT

A method of characterizing responses from a plurality of chemical sensors to detect a chemical agent without interference or false alarms from other chemical present, comprising providing a plurality of different sensors, building a library of known responds of each of the plurality of different sensors to the chemical agent, and using a continuous inference network to model the plurality of different sensors based on the library of known response and relationship between the response of the plurality of different sensors and the chemical agent.

### CROSS-REFERENCE TO RELATED APPLICATIONS

This application claims priority to U.S. Provisional Patent Application No. 60/723,525, filed Oct. 4, 2005, herein incorporated by reference in their entirety.

### GRANT REFERENCE

Work related to the invention disclosed in this application was performed under U.S. Marine Corps., Contract No. M67004-99-D-0037/MU-60. The government may have certain rights in this invention.

### BACKGROUND OF THE INVENTION

Typical chemical detection uses one or more sensors to measure some atomic spectral aspect(s) of the molecule of interest, determine a threshold above which a “detection” is assigned, and then repeat the process for every chemical of interest. The limitation of this standard approach is that there is a large number of chemicals and chemical mixtures that also trigger detections in this manner. Thus, for sensing chemicals in uncontrolled environments, many false alarms occur naturally. It is impractical to compare detections from millions of possible interferants, so false alarms are a common occurrence. Therefore, problems remain with chemical detection.

Although seemingly unrelated to chemical detection, to one skilled in the art who does not have the benefit of this disclosure, a Continuous Inference Network (CINET) is a technique for fusion of information in computing software. It allows continuous blending of information with varying confidences using fuzzy logic. This logic follows human expertise in that combinations of various information inputs may have unique logic applied relative to the situation, or context of the recognition decision. One example of a CINET is described in U.S. Pat. No. 5,642,467 to Stover et al., herein incorporated by reference in its entirety, which discloses a controller for directing the actions of an autonomous device in response to the existence or actions of objects using a program for fusing physical world data and inferred property confidence factors into representational instances.

### BRIEF SUMMARY OF THE INVENTION

Therefore, it is a primary object, feature, or advantage of the present invention to improve upon the state of the art.

It is a further object, feature, or advantage of the present invention to characterize the responses of multiple and diverse chemical sensors to construct sensor models and use the sensor models to detect specific chemical agents without interference from or false alarms from other chemicals that may be present.

A still further object, feature, or advantage of the present invention is to use a CINET with embedded sensor models in chemical detection.

One or more of these and/or other objects, features, or advantages of the present invention will become apparent from the specification and claims that follow.

One purpose of this invention is to characterize the responses of several diverse chemical sensors as a function of chemical and concentration for the chemical agents of interest, and to use the sensor response models to uniquely detect specific agents without interference or false alarms from other chemicals that might be present. The technique allows the use of available sensor technology, fuses the sensor outputs in a novel way using a CINET with embedded sensor models, and thus enhances false alarm rejection without reducing detection sensitivity. This invention provides for modeling and employing various chemical sensors together to reduce false alarms.

According to one aspect of the present invention, a method of characterizing responses from a plurality of chemical sensors to detect a chemical agent without interference or false alarms from other chemicals present is provided. The method includes providing a plurality of different sensors, building a library of known responses of each of the plurality of different sensors to the chemical agent, and using a continuous inference network to model the plurality of different sensors based on the library of known responses and the relationship between the responses of the plurality of different sensors and the chemical agent. The chemical agents may be associated with a weapon of mass destruction. The sensors can be of various types including, without limitation, flame photometric detection sensors, surface acoustic wave sensors, ion mobility spectroscopy sensors, and photo ionization detection sensors.

According to another aspect of the present invention, a method of applying a continuous inference network to determine presence of a chemical agent is provided. The method includes sensing chemical properties with a plurality of different sensors, applying the sensed chemical properties as inputs to the continuous inference network, and outputting an alert condition and a confidence level for the chemical agent from the continuous inference network.

### BRIEF DESCRIPTION OF THE DRAWINGS

FIG. 1A illustrates a comparison of models for an FPD, SAW, and JMS sensors for the chemical GB (Sarin).

FIG. 1B illustrates a comparison of models for an FPD, SAW, and JMS sensors for the chemical HO (Mustard).

FIG. 2 illustrates one embodiment of a CINET with chemical sensor models and floating blends to reject false alarms from chemical interferants.

FIG. 3 illustrates one embodiment of a method for characterizing responses from chemical sensors to detect a chemical agent without interference or false alarms from other chemicals present.

FIG. 4 illustrates one embodiment of a method for applying a CINET to determine the presence of a chemical agent.

### DETAILED DESCRIPTION OF THE PREFERRED EMBODIMENT

The present invention is of particular interest with respect to trace detection problem for chemicals used as weapons of mass destruction, but the same procedure applies to explosives detection, illegal drug detection, and even disease detection through natural biological chemical production. In such cases, of interest is a small (trace) amount of some chemical within a large population of various chemicals.

In a first step, we choose two or more fundamentally different sensor technologies that rely of different physical principles thus providing nearly orthogonal independent metrics of the atomic spectra of the chemical of interest. Diverse sensor examples are: flame photometric detection (FPD) which use burning in hydrogen to detect specific atoms via spectroscopy, surface acoustic wave (SAW) which measure chemical mass adsorption rates through surface polymer resonance shift, ion mobility spectroscopy (IMS) which measure electric mobility of ions against a constant gas flow, or photo ionization detection (PID) which use intense ultra violet light to ionize molecules and produce a measurable current. These sensors ultimately produce, directly or as a result of an algorithm, a “level” which varies with chemical and concentration detected.

Next, we characterize for each chemical of interest, each sensor's output level response as a function of concentration of that chemical. This builds a library of known responses to chemicals for each sensor. Our practical assumption here is that the list of chemicals we are interested in is much less than the number of possible chemical interferants.

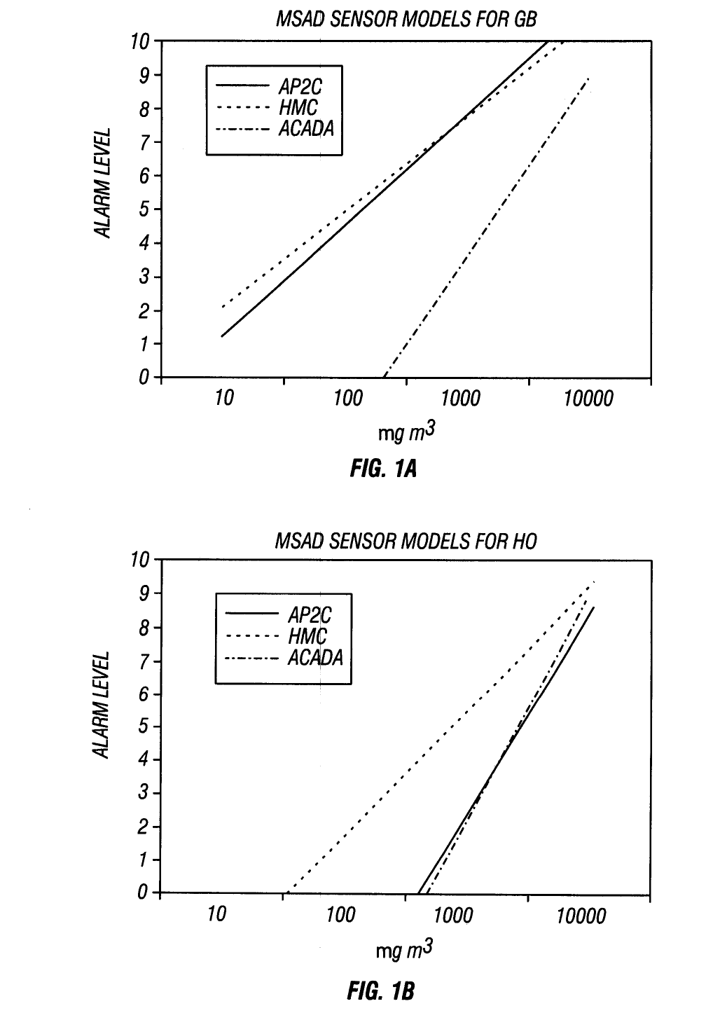

FIG. 1A and FIG. 1B illustrate that diverse sensors respond differently as a function of concentration and chemical molecule. In FIG. 1A, a concentration model for GB (Sarin) using an FPD (an AP2C sensor), a SAW (an HMC sensor), and an IMCS (an ACADA) sensor is shown. In FIG. 1B, a concentration model is provided for HO (Mustard) with the same three types of diverse sensors. Note that the diverse sensors respond differently as a function of concentration and chemical molecule. This was found to be the case for a wide range of known chemical interferants. In other words, no two chemicals produce the same three sensor response curves. Therefore, we can reject chemicals where the sensors' relative alarm levels do not match the models for the assumed concentration.

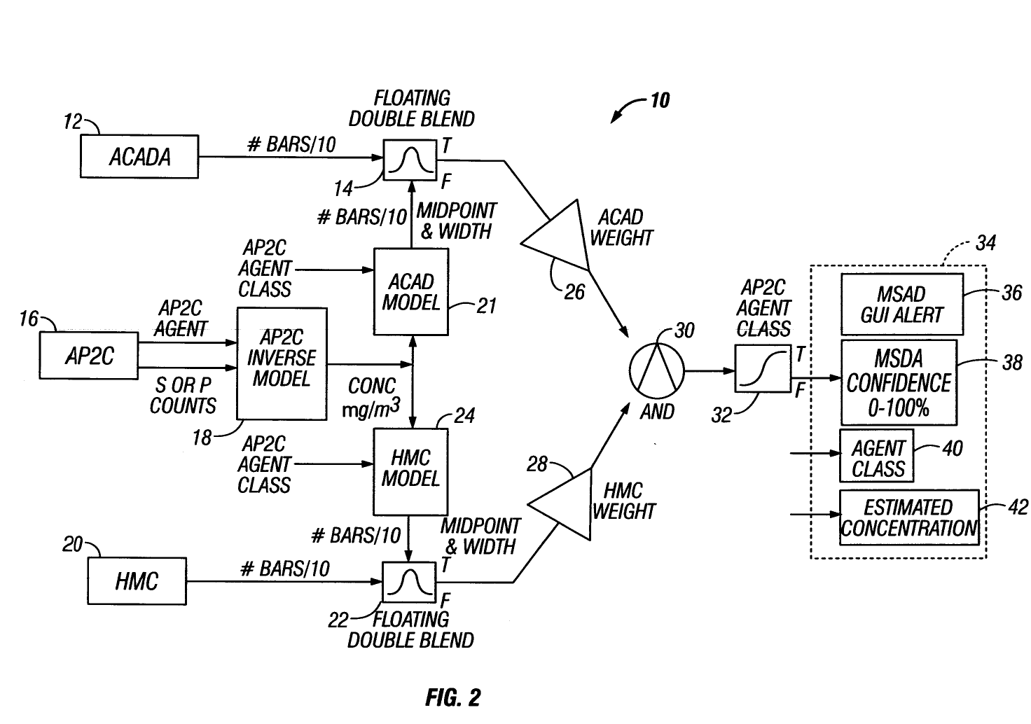

FIG. 2 shows a CINET which implements one embodiment of the invention. It differs from other CINET architectures in that the sensor models are embedded and the blend functions “float” according to the model outputs. In FIG. 2, the CINET 10 uses a plurality of sensors including an Automatic Chemical Agent Detection and Alarm (ACADA) sensor 12, an AP2C sensor 16, and an HMC sensor 20. An AP2C is an example of a flame spectrophotometer for gas detection and is available from Proengin.

The AP2C sensor 16 extracts the concentration hypothesis base on the counts from its photo multiplier tube (PMT). The PMT counts are quantum events directly proportional to concentration of the atomic elements excited in the hydrogen flame. An AP2C inverse model 18 is shown which provides the concentration. An ACAD model 21 is provided as well as an HMC model 24. A floating double blend function 14 is used to combine output from the ACADA sensor 12 and the ACAD model 21. A floating double blend function 22 is used to combine output from the HMC sensor 20 and the HMC model 24. An ACAD weight 26 is applied to the output of the floating double blend function 14 and an HMC weight is applied to the output of the other floating double blend function 22 when the outputs of the functions are combined using an AND 30. The double blend functions may be tuned to be true at the predicted alarm level and blending off to false in either direction. The width of each blend is adjusted along with the relative weights for the sensors to control the desired behavior of the CINET. The output is then provided to an AP2C agent class 32 and appropriate outputs 34 are provided. The outputs 34 may include an alert 36, a confidence level 38, an agent class 40, and an estimated concentration 42. Knowing the confidence level as well as an estimated concentration is important in being able to assess what action should be taken. By way of example, for the inference that we have chemical GB at concentration K, we predict the sensor alarm levels for the SAW (HMC) and IMS (ACADA) sensors and create a double blend function tuned to be true at the predicted alarm level and blending off to false in either direction. The width of the blend is adjusted along with the relative weights for the sensors to control the desired behavior of the CINET.

One advantage provided is that the sensor model and CINET are only needed for each chemical of interest and sensor used. This allows current and future sensors to be used in various combinations to reject false alarms without requiring modification of the sensors. The CINET which fuses the sensor responses is laid out in advance and if a particular sensor is used, the corresponding part of the CINET is “turned on” by the associated weights and logic. This allows the CINET's to be run remotely from the sensors and dynamically upgraded to interpret new chemical threats and situations. The sensor models for each chemical can be stored in the sensor as a data “manifest” or dynamically updated from networked libraries. This creates a static market for sensors, but allows rapid updating of the overall CINET detection performance through networked information.

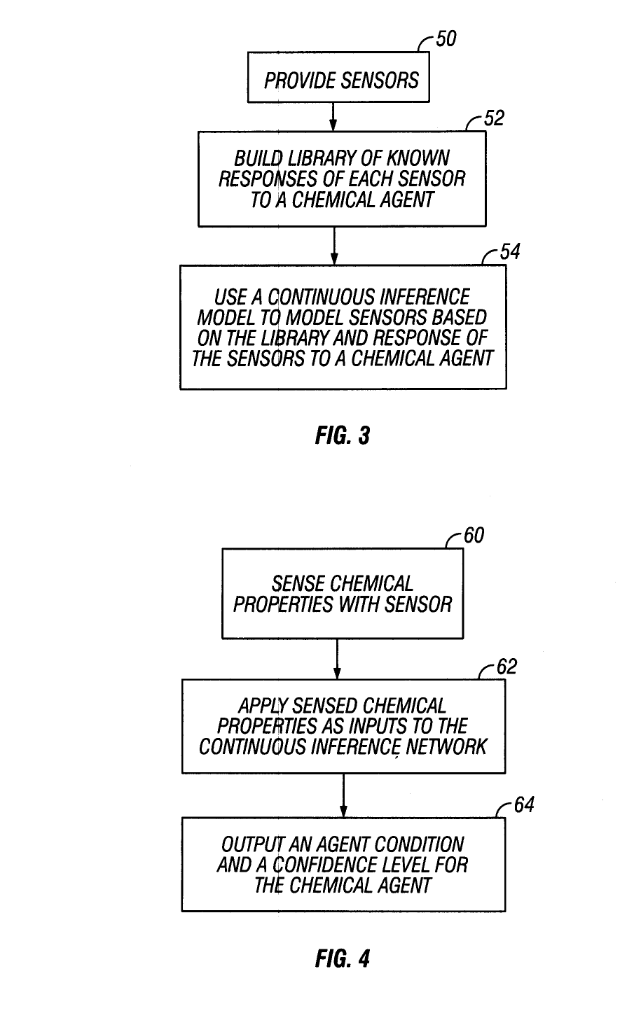

FIG. 3 illustrates one embodiment of a method for characterizing responses from chemical sensors to detect a chemical agent without interference or false alarms from other chemicals present. In step 50, sensors are provided. In step 52, a library of known responses to a chemical for each sensor is built. In step 54 a continuous inference model is used to model sensors based on the library and response of the sensors to a chemical agent.

FIG. 4 illustrates one embodiment of a method for applying a CINET to determine the presence of a chemical agent. In step 60, chemical properties are sensed with a sensor. Next, step 62 provides for applying sensed chemical properties as inputs to a continuous inference network. In step 64, an agent condition and confidence level are output for the chemical agent.

The procedure described in this invention can be applied to any type of sensor. The sensor's response itself is used as a feature in a space that consists of a number of diverse sensors. Therefore a given sensor's lack of response may be as important as its strong response for a given chemical. This approach may be used for biological chemical sensing where the molecules are very long and complex, yet constructed from a small number of amino or nucleic acids in various combinations. Optical markers which bind to parts of these biological molecules may provide varying “levels” of response, which differs with each biological marker and concentration. Following the procedure described, CINETs with dynamic concentration models and blends could be used to isolate and detect biological material using large numbers of biological sensors and their collective pattern of responses.

Of course, the present invention contemplates numerous variations in addition to the exemplary embodiments disclosed herein. For example, numerous other sensor technologies may be used, any number of chemical agents may be of interested, and various other types of problems may be addressed. These and other variations and alternatives are within the broad spirit and scope of the invention.

What is claimed is:

1. A method of characterizing responses from a plurality of chemical sensors to detect a chemical agent without interference or false alarms from other chemical present, comprising:
providing a plurality of different sensors;
building a library of known responds of each of the plurality of different sensors to the chemical agent;
using a continuous inference network to model the plurality of different sensors based on the library of known response and relationship between the response of the plurality of different sensors and the chemical agent.

2. The method of claim 1 wherein the chemical agent is associated with a weapon of mass destruction.

3. The method of claim 1 wherein the model of the plurality of different sensors includes a floating blend function.

4. The method of claim 1 wherein one of the plurality of different sensors is a flame photometric detection sensor.

5. The method of claim 1 wherein one of the plurality of different sensors is a surface acoustic wave sensor.

6. The method of claim 1 wherein one of the plurality of different sensors is an ion mobility spectroscopy sensor.

7. The method of claim 1 wherein one of the plurality of different sensors is a photo ionization detection sensor.

8. The method of claim 1 further comprising applying the continuous inference network to detect presence of the chemical agent.

9. The method of claim 1 wherein one of the plurality of different sensors is a flame photometric detection sensor, one of the plurality of different sensors is a surface acoustic wave sensor, and one of the plurality of different sensors is an ion mobility spectroscopy sensor.

10. The method of claim 9 wherein the model of the plurality of different sensors includes a floating blend function.

11. The method of claim 10 further comprising applying the continuous inference network to detect presence of the chemical agent.

12. A method of applying a continuous inference network to determine presence of a chemical agent comprising:
sensing chemical properties with a plurality of different sensors;
applying the sensed chemical properties as inputs to the continuous inference network;
outputting an alert condition and a confidence level for the chemical agent from the continuous inference network.

13. The method of claim 12 further comprising outputting an estimated concentration level for the chemical agent.

14. The method of claim 12 wherein one of the plurality of different sensors is a flame photometric detection sensor.

15. The method of claim 12 wherein one of the plurality of different sensors is a surface acoustic wave sensor.

16. The method of claim 12 wherein one of the plurality of different sensors is an ion mobility spectroscopy sensor.

17. The method of claim 12 wherein one of the plurality of different sensors is a photo ionization detection sensor.

18. The method of claim 12 wherein the chemical agent is mustard gas.

19. The method of claim 12 wherein the chemical agent is sarin gas.

20. The method of claim 12 wherein one of the plurality of different sensors is a flame photometric detection sensor, one of the plurality of different sensors is a surface acoustic wave sensor, and one of the plurality of different sensors is an ion mobility spectroscopy sensor.
